(19) United States
(12) Patent Application Publication
(10) Pub. No.: US 2004/0267256 A1
(43) Pub. Date: Dec. 30, 2004

(54) Compound lesion alignment device

(76) Inventors: Robert J. Garabedian, Mountain View, CA (US); Amy C. Kelly, San Francisco, CA (US); Steven K. Landreville, Mountain View, CA (US)

Correspondence Address: Bingham McCutchen LLP, Suite 1800, Three Embarcadero Center, San Francisco, CA, 94111-4067, US

(21) Appl. No.: 10/606,250

(22) Filed: Jun. 24, 2003

(51) Int. Cl. A61B 18/14

(52) U.S. Cl. 606/41

(57) ABSTRACT

A medical probe assembly and method for ablating tissue using radio frequency energy is provided. Included in the medical probe assembly is an ablation probe and an alignment device. The alignment device comprises a surface and plurality of apertures through which the ablation probe can be guided into the target region of the patient. The apertures may be uniformly or non-uniformly spaced and parallel or non-parallel from each other. The apertures may be indexed from each other in a two dimensional plane. By adding one or more bosses or recesses to the apertures, the apertures may indexed from each other in a three dimensional space and provides an improved system and method for accurately creating compound lesions on tumors. Furthermore, by adding removable inserts to the recesses, the depth of the recess may be adjustable.

### FIELD OF THE INVENTION

The field of the invention relates generally to the use of ablation probes for the treatment of tissue, and more particularly, RF ablation probes for the treatment of tumors.

### BACKGROUND OF THE INVENTION

The delivery of radio frequency (RF) energy to target regions within tissue is known for a variety of purposes of particular interest to the present invention. In one particular application, RF energy may be delivered to diseased regions (e.g., tumors) in target tissue for the purpose of tissue necrosis.

One method for RF ablation uses a single needle electrode, which when attached to a RF generator, emits RF energy from the exposed, uninsulated portion of the electrode. This energy translates into ion agitation, which is converted into heat and induces cellular death via coagulation necrosis. By varying the power output and the type of electrical waveform, it is possible to control the extent of heating, and thus, the resulting ablation. The diameter of tissue coagulation from a single electrode, however, has been limited by heat dispersion.

Another method for ablation utilizes multiple needle electrodes, which have been designed for the treatment and necrosis of tumors in the liver and other solid tissues. PCT application WO 96/29946 and U.S. Pat. No. 6,379,353 disclose such probes. In U.S. Pat. No. 6,379,353, a probe system comprises a cannula having a needle electrode array reciprocably mounted therein. The individual electrodes within the array have spring memory, so that they assume a radially outward, arcuate configuration as they are advanced distally from the cannula. In general, a multiple electrode array creates a larger lesion than that created by a single needle electrode.

When performing an ablation on a tumor, the general rule is to select an array that has a diameter that will produce a 1 cm margin of ablated tissue around the periphery of the actual tumor. For example, for a 1 cm tumor, the appropriate array diameter would be 3.0 cm. Unfortunately, many of the tumors currently treated are larger than 1 cm in diameter. Often, the tumor is larger than the largest available array device (4.0 cm) currently on the market, the LaVeen probe offered by Boston Scientific. In theory, the largest tumor size that the 4.0 cm device can treat on a single ablation is 2.0 cm (4.0 cm device−2.0 cm margin=2.0 cm tumor). When treating tumors that are larger than 2.0 cm, generally, an ablation is performed and then the array is repositioned around the initial ablation. This process is continued until the overlapping ablations create a 1 cm margin over the tumor.

One difficulty experienced with creating a compound lesion is the reduced ultrasonic image visualization caused by an echogenic cloud from the initial ablation. Physicians must estimate the initial location and depth and then reposition the array for subsequent overlapping ablations. This process proves to be challenging because of poor imaging quality. Moreover, the individual ablation devices will generally not be steerable and capable of being redirected within the tissue, so there are few options for correcting the configuration after the needles have first penetrated into the tissue.

Thus, there is a need to provide improved systems and methods for accurately creating compound lesions on tumors.

### SUMMARY OF THE INVENTION

In accordance with a first aspect of the present inventions, a tissue ablation system is provided. The tissue ablation system comprises one or more ablation probes. In the preferred embodiment, the ablation probe(s) utilize radio frequency (RF) energy, but it can also utilize other types of energy, such as laser energy. The tissue ablation system further comprises an alignment device configured to be fixed relative to targeted tissue, e.g., a tumor. In the preferred embodiment, the alignment device can be conveniently adhered to the patient, but other types of suitable means can be used to affixed the alignment device relative to the targeted tissue. The alignment device can be any shape, including a customized shape, but in the preferred embodiment, it is disk-shaped.

The alignment device comprises a surface and a plurality of apertures through which the ablation probe(s) can be guided. The apertures can be spaced apart along the surface in any of a variety of configurations. For example, the spacing between the apertures can either be fixed or adjustable. The spacing between the apertures can be uniform or non-uniform. The axes of the apertures can be parallel or non-parallel to each other. For example, if the apertures are parallel, the ablation probes(s) can be aligned in a Cartesian coordinate system. If the apertures are non-parallel, the ablation probe(s) can be aligned in an angular coordinate system. In one preferred embodiment, the apertures comprise a central aperture and remaining apertures that are placed in a plurality of concentric rings around the central aperture.

Thus, it can be appreciated that the apertures can be indexed from each other in a two-dimensional plane. Optionally, the alignment device can comprise one or more bosses or recesses associated with a respective one or more of the plurality of apertures, wherein the boss(es) limits and recess(es) increase the distance that the ablation probe(s) can be guided through the aperture(s). If a plurality of boss(es) is provided, the bosses can have differing lengths. Likewise, if a plurality of recesses are provided, the recesses can have variable depths. The boss(es) can either be permanently mounted or removably mounted to the aperture(s). The recess(es) can also be “filled” with insert(s). Thus, it can be appreciated that the boss(es) and recess(es) allow the apertures to be indexed from each other in three-dimensional space.

In accordance with a second aspect of the present inventions, a method for performing compound ablation in the body of a patient is provided. The method comprises affixing an alignment device relative to target tissue, such as, e.g., a tumor. The alignment device can be affixed using any suitable means, e.g., by adhering the alignment device to the skin of the patient. The method further comprises guiding an ablation probe within a first aperture in the alignment device to place the ablation probe adjacent the targeted tissue in a first region. For example, the ablation probe can be placed in contact with the targeted tissue (e.g., by embedding it) or placed a relative short distance from the targeted tissue. The ablation probe can be placed adjacent the targeted tissue using any suitable means. For example, the ablation probe can be introduced into the patient's body percutaneously, laparoscopically, or through a surgical opening.

The method further comprises operating the ablation probe (e.g., using RF or laser energy) to create a first lesion in the first region. The method further comprises guiding the ablation probe within a second different aperture in the alignment device to place the ablation probe adjacent the targeted tissue in a second region, and operating the ablation probe again to create a second lesion in the second region. In addition, the ablation device can be guided to a different depth within the first aperture in the alignment device to place the ablation probe adjacent the targeted tissue in a second region, and operating the ablation probe to create a second lesion in the second region. The ablation probe may be removed completely from the first aperture prior to guiding it within the second aperture. Alternatively, the ablation probe may be moved from the first aperture to the second aperture without completely removing the ablation probe, e.g., by laterally guiding the ablation probe along a guiding slot between the first and second apertures. In any event, alternate guiding and operating of the ablation probe can be performed for a plurality of regions until the entire target tissue is ablated.

The ablation probe can be guided within the first and second apertures in parallel directions, e.g., to align the ablation probe in a Cartesian coordinate system, or can be guided within the first and second apertures in non-parallel directions, e.g., to align the ablation probe in an angular coordinate system. The alignment device can optionally comprise a boss or a recess associated with the first aperture, in which case, the method can comprise limiting a distance that the ablation probe is guided within the first aperture by abutting a portion of the ablation probe against the boss or recess.

In accordance with a third aspect of the present invention, another method of performing a compound ablation in the body of a patient is provided. The method comprises affixing an alignment device relative to target tissue, such as, e.g., a tumor. The alignment device can be affixed using any suitable means, e.g., by adhering the alignment device to the skin of the patient. The method further comprises guiding a plurality of ablation probes within a respective plurality of apertures in the alignment device to place the ablation probes adjacent the targeted tissue in a plurality of regions. For example, the ablation probes can be placed in contact with the targeted tissue (e.g., by embedding them) or placed a relative short distance from the targeted tissue. The ablation probes can be placed adjacent the targeted tissue using any suitable means. For example, the ablation probes can be introduced into the patient's body percutaneously, laparoscopically, or through a surgical opening.

The ablation probes can be guided within the apertures in parallel directions, e.g., to align the ablation probes in a Cartesian coordinate system, or can be guided within the apertures in non-parallel directions, e.g., to align the ablation probes in an angular coordinate system. The alignment device can optionally comprise one or more bosses or recesses associated with one or more of the apertures, in which case, the method can comprise limiting a distance that one or more of the ablation probes is guided within the aperture(s) by abutting a portion of the ablation probe(s) against the boss(es) or recess(es). If a plurality of bosses or recesses are provided, the bosses or recesses can have differing lengths.

The method further comprises operating the ablation probes (e.g., using RF or laser energy) to create a plurality of lesions within the plurality of regions. The ablation probes can either be operated in a unipolar mode or a bipolar mode (e.g., by conveying RF energy between two ablation probes).

In accordance with a fourth aspect of the present inventions, an alignment device for one or more ablation probes is provided. In the preferred embodiment, the alignment device can be conveniently adhered to the patient, but other types of suitable means can be used to affixed the alignment device relative to the targeted tissue. The alignment device can be any shape, including a customized shape, but in the preferred embodiment, it is disk-shaped. The alignment device comprises a surface and a plurality of apertures through which the ablation probe(s) can be guided. The apertures can be spaced apart along the surface in any of a variety of configurations, as previously described.

The alignment device further comprises one or more bosses and/or recesses associated with a respective one or more of the plurality of apertures, wherein the boss(es) or recess(es) limits the distance that the ablation probe(s) can be guided through the aperture(s). If a plurality of boss(es) or recess(es) is provided, the bosses or recesses can have differing lengths. If boss(es) are provided, the boss(es) can either be permanently mounted or removably mounted to the aperture(s). If recess(es) are provided, the recess may be associated with an insert that decreases the depth of the recess. Thus, it can be appreciated that the boss(es) and/or recess(es) allow the apertures to be indexed from each other in three-dimensional space.

### BRIEF DESCRIPTION OF THE DRAWINGS

The drawings illustrate the design and utility of a preferred embodiment of the present invention, in which similar elements are referred to by common reference numerals. In order to better appreciate the advantages and objects of the present invention, reference should be made to the accompanying drawings that illustrate this preferred embodiment. However, the drawings depict only one embodiment of the invention, and should not be taken as limiting its scope. With this caveat, the invention will be described and explained with additional specificity and detail through the use of the accompanying drawings in which:

FIG. 1 is a perspective view of a tissue ablation system constructed in accordance with one preferred embodiment of the present invention, wherein a single probe assembly is particularly shown used with the alignment device of FIG. 4;

FIG. 2 is a perspective view of an ablation probe assembly used in the tissue ablation system of FIG. 1, wherein a needle electrode array is particularly shown retracted;

FIG. 3 is a perspective view of the ablation probe assembly used in the tissue ablation system of FIG. 1, wherein a needle electrode array is particularly shown deployed;

FIG. 4 is a perspective view of a first embodiment of an alignment device that can used in the tissue ablation system of FIG. 1;

FIG. 5 is a cross-sectional view of the alignment device of FIG. 4;

FIG. 6 is a cross-sectional view of a second embodiment of an alignment device that can be used in the tissue ablation system of FIG. 1;

FIG. 7 is a cross-sectional view of a third embodiment of an alignment device that can be used in the tissue ablation system of FIG. 1;

FIG. 8 is a perspective view of a tissue ablation system constructed in accordance with another preferred embodiment of the present invention, wherein multiple probe assemblies are particularly shown used with the alignment device of FIG. 7;

FIG. 9 is a cross-sectional view of a fourth embodiment of an alignment device that can be used in the tissue ablation system of FIG. 1;

FIG. 10 is a cross-sectional view of a fifth embodiment of an alignment device that can be used in the tissue ablation system of FIG. 1;

FIG. 11 is a cross-sectional view of a sixth embodiment of an alignment device that can be used in the tissue ablation system of FIG. 1;

FIG. 12 is a cross-sectional view of a seventh embodiment of an alignment device that can be used in the tissue ablation system of FIG. 1;

FIG. 13 is a cross-sectional view of an eighth embodiment of an alignment device that can be used in the tissue ablation system of FIG. 1;

FIGS. 14-17 are perspective views illustrating one preferred method of using the tissue ablation system of FIG. 1 to ablate a treatment region within tissue of a patient;

FIG. 18 is a perspective view illustrating another preferred method of using the tissue ablation system of FIG. 1 to ablate the treatment region; and

FIG. 19 is a perspective view illustrating a preferred method of using the tissue ablation system of FIG. 8 to ablate the treatment region.

### DETAILED DESCRIPTION OF THE PREFERRED EMBODIMENTS

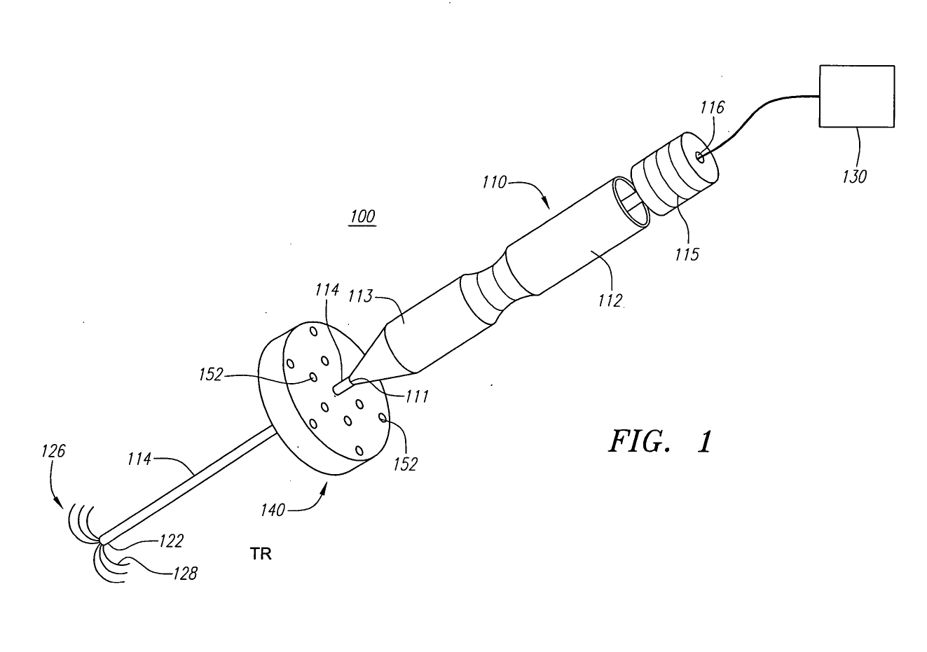

FIG. 1 illustrates a tissue ablation system 100 constructed in accordance with a preferred embodiment of the present invention. The tissue ablation system 100 generally comprises an ablation probe assembly 110, which is configured for introduction into the body of a patient to ablate target tissue such as a tumor, a radio frequency (RF) generator 130 configured for supplying RF energy to the probe assembly 110 in a controlled manner, and an alignment device 140 configured for ensuring accurate positioning of the ablation probe assembly 110 relative to the target tissue. In the illustrated embodiment, only one probe assembly 110 is shown. As will be described in further detail below, however, multiple probe assemblies 110 can be connected to the RF generator 130 and simultaneously associated with the alignment device 140, depending upon the specific ablation procedure that the physician selects.

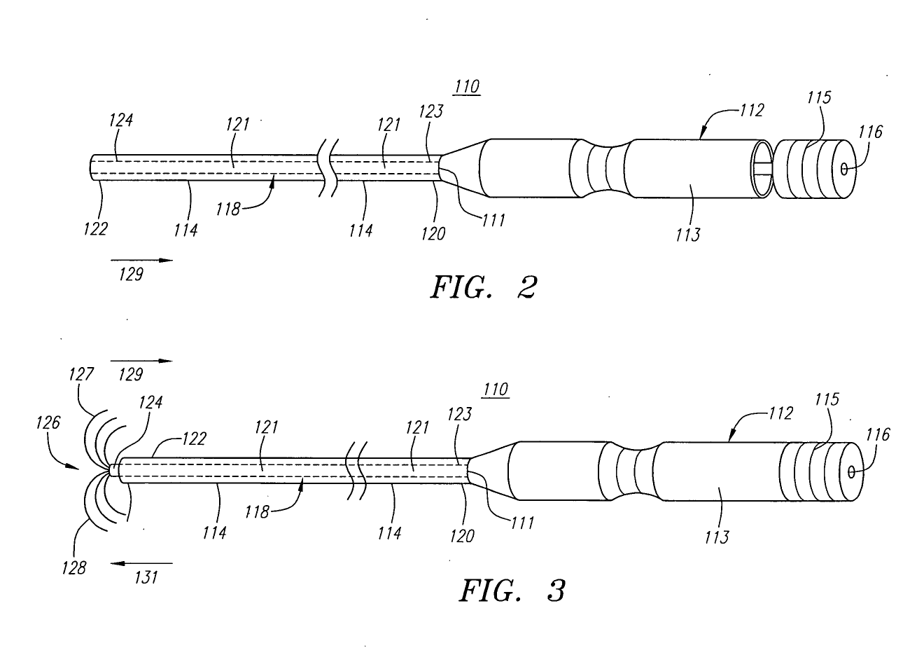

Referring further to FIGS. 2 and 3, the probe assembly 110 generally comprises a handle assembly 112, an elongated cannula 114, and an inner probe 118 (shown in phantom) slideably disposed within the cannula 114. As will be described in further detail below, the cannula 114 serves to deliver the active portion of the inner probe 118 to the target tissue. The cannula 114 has a proximal end 120, a distal end 122, and a central lumen (not shown) extending through the cannula 114 between the proximal end 120 and the distal end 122. The cannula 114 may be rigid, semi-rigid, or flexible depending upon the designed means for introducing the cannula 114 to the target tissue. The cannula 114 is composed of a suitable material, such as plastic or metal, and has a suitable length, typically in the range of 5 cm to 30 cm, preferably from 10 cm to 20 cm. The cannula 114 has an outside diameter consistent with its intended use, typically being from 1 mm to 5 mm, usually from 1.3 mm to 4 mm. The cannula 114 has an inner diameter in the range of 0.7 mm to 4 mm, preferably from 1 mm to 3.5 mm.

The inner probe 118 comprises a reciprocating shaft 121 having a proximal end 123 and a distal end 124, and an array 126 of tissue penetrating needle electrodes 128 extending from the distal end 124 of the shaft 121. Like the cannula 114, the shaft 121 is composed of a suitable material, such as plastic or metal. The electrode array 126 can be mounted anywhere on the shaft 121. However, the electrodes 128 will typically be fastened to the shaft 121 at its distal end 124, though the individual electrodes 128 can extend up to its proximal end 123. Each of the needle electrodes 128 is a small diameter metal element, which can penetrate into tissue as it is advanced through tissue.

As illustrated in FIG. 2, longitudinal translation of the shaft 121 in the proximal direction 129 relative to the cannula 114, retracts the electrode array (not shown) into the distal end 122 of the cannula 114. When retracted within the cannula 114, the electrode array 126 (shown in FIG. 3) is placed in a radially collapsed configuration, and each needle electrode 128 is constrained and held in a generally axially aligned position within the cannula 114 to facilitate its introduction into tissue. The probe assembly 110 optionally includes a core member (not shown) mounted on the distal end 124 of the shaft 121 and disposed within the center of the needle electrode array 126. In this manner, substantially equal circumferential spacing between adjacent needle electrodes 128 is maintained when the array is retracted within the central lumen.

As shown in FIG. 3, longitudinal translation of the shaft 121 in the disial direction 131 relative to the cannula 114 deploys the electrode array 126 out of the distal end 122 of the cannula 114. As will be described in further detail, manipulation of the handle assembly 112 will cause the shaft 121 to longitudinally translate to alternately retract and deploy the electrode array 126.

When deployed from the cannula 114, the electrode array 126 is placed in a three-dimensional configuration that usually defines a generally spherical or ellipsoidal volume having a periphery with a maximum radius in the range of 0.5 cm to 4 cm. The needle electrodes 128 are resilient and pre-shaped to assume a desired configuration when advanced into tissue. In the illustrated embodiment, the needle electrodes 128 diverge radially outwardly from the cannula 114 in a uniform pattern, i.e., with the spacing between adjacent needle electrodes 128 diverging in a substantially uniform pattern or symmetric pattern or both. In the illustrated embodiment, the needle electrodes 128 evert proximally, so that they face partially or fully in the proximal direction 129 when fully deployed. In exemplary embodiments, pairs of adjacent needle electrodes 128 can be spaced from each other in similar or identical, repeated patterns that can be symmetrically positioned about an axis of the shaft 121. It will be appreciated by one of ordinary skill in the art that a wide variety of patterns can be used to uniformly cover the region to be treated. It should be noted that a total of six needle electrodes 128 are illustrated in FIGS. 1 and 3. Additional needle electrodes 128 can be added in the spaces between the illustrated electrodes 128, with the maximum number of needle electrodes 128 determined by the electrode width and total circumferential distance available. Thus, the needle electrodes 128 could be quite tightly packed.

Each electrode 128 is preferably composed of a single wire that is formed from resilient conductive metals having a suitable shape memory. Many different metals such as stainless steel, nickel-titanium alloys, nickel-chromium alloys, and spring steel alloys can be used for this purpose. The wires may have circular or non-circular cross-sections, but preferably have rectilinear cross-sections. When constructed in this fashion, the needle electrodes 128 are generally stiffer in the transverse direction and more flexible in the radial direction. The circumferential alignment of the needle electrodes 128 within the cannula 114 can be enhanced by increasing transverse stiffness. Exemplary needle electrodes will have a width in the circumferential direction in the range of 0.2 mm to 0.6 mm, preferably from 0.35 mm to 0.40 mm, and a thickness, in the radial direction, in the range of 0.05 mm to 0.3 mm, preferably from 0.1 mm to 0.2 mm.

The distal ends 127 of the needle electrodes 128 may be honed or sharpened to facilitate their ability to penetrate tissue. The distal ends 127 of these needle electrodes 128 may be hardened using conventional heat treatment or other metallurgical processes. The needle electrodes 128 may be partially covered with insulation, although they will be at least partially free from insulation over their distal portions 127. The proximal ends 127 of the needle electrodes 128 may be directly coupled to the proximal end of the shaft 121, or alternatively, may be indirectly coupled thereto via other intermediate conductors such as RF wires. Optionally, the shaft 121 and any component between the shaft 121 and the needle electrodes 128 are composed of an electrically conductive material, such as stainless steel, and may, therefore, conveniently serve as intermediate electrical conductors.

Referring still to FIGS. 2 and 3, the steerable handle assembly 110 is mounted to the cannula 114 and inner probe shaft 121 and serves to conveniently allow the physician to alternately deploy and retract the electrode array 126. Specifically, the handle assembly 110 comprises distal and proximal handle members 113 and 115 that are slidingly engaged with each other. The distal handle member 113 is mounted to the proximal end 120 of the cannula 114, and the proximal handle member 115 is mounted to the proximal end 123 of the inner probe shaft 121. The proximal handle member 115 also comprises an electrical connector 116, which electrically couples the RF generator 130 to the proximal ends of the needle electrodes 128 (or alternatively, the intermediate conductors) extending through the inner probe shaft 121. The handle assembly 110 can be composed of any suitable rigid material, such as e.g., metal, plastic, or the like.

In the illustrated embodiment, the RF current is delivered to the electrode array 126 in a mono-polar fashion. Therefore, the current will pass through the electrode array 126 and into the target tissue, thus inducing necrosis in the tissue. To this end, the electrode array 126 is configured to concentrate the energy flux in order to have an injurious effect on tissue. However, there is a dispersive electrode (not shown) which is located remotely from the electrode array 126, and has a sufficiently large area—typically 130 cm² for an adult—so that the current density is low and non-injurious to surrounding tissue. In the illustrated embodiment, the dispersive electrode may be attached externally to the patient, using a contact pad placed on the patient's skin. In a mono-polar arrangement, the needle electrodes 128 are bundled together with their proximal portions 127 having only a single layer of insulation over the entire bundle.

Alternatively, the RF current is delivered to the electrode array 126 in a bipolar fashion, which means that current will pass between “positive” and “negative” electrodes 128 within the array 126. In a bipolar arrangement, the positive and negative needle electrodes 128 will be insulated from each other in any regions where they would or could be in contact with each other during the power delivery phase. As will be described in further detail below, RF current can also pass between electrode arrays of two or more probe assemblies in a bipolar fashion.

Further details regarding needle electrode array-type probe arrangements are disclosed in U.S. Pat. No. 6,379,353, entitled “Apparatus and Method for Treating Tissue with Multiple Electrodes,” which is expressly incorporated herein by reference.

The probe assembly 110 may optionally have active cooling functionality, in which case, a heat sink (not shown) can be mounted within the distal end 125 of the shaft 121 in thermal communication with the electrode array 126, and cooling and return lumens (not shown) can extend through the shaft 121 in fluid communication with the heat sink to draw thermal energy away back to the proximal end 124 of the shaft 121. A pump assembly (not shown) can be provided to convey a cooling medium through the cooling lumen to the heat sink, and to pump the heated cooling medium away from the heat sink and back through the return lumen. Further details regarding active cooling of the electrode array 126 are disclosed in co-pending U.S. application Ser. No. ______ (Bingham McCutchen Docket No. 24728-7011), which is expressly incorporated herein by reference.

Referring back to FIG. 1, as previously noted, the RF generator 130 is electrically connected, via the generator connector 116, to the handle assembly 112, which is directly or indirectly electrically coupled to the electrode array 126. The RF generator 130 is a conventional RF power supply that operates at a frequency in the range of 200 KHz to 1.25 MHz, with a conventional sinusoidal or non-sinusoidal wave form. Such power supplies are available from many commercial suppliers, such as Valleylab, Aspen, and Bovie. Most general purpose electro-surgical power supplies, however, operate at higher voltages and powers than would normally be necessary or suitable for controlled tissue ablation.

Thus, such power supplies would usually be operated at the lower ends of their voltage and power capabilities. More suitable power supplies will be capable of supplying an ablation current at a relatively low voltage, typically below 150V (peak-to-peak), usually being from 50V to 100V. The power will usually be from 20 W to 200 W, usually having a sine wave form, although other wave forms would also be acceptable. Power supplies capable of operating within these ranges are available from commercial vendors, such as RadioTherapeutics of San Jose, Calif., which markets these power supplies under the trademarks RF2000™ (100 W) and RF3000™ (200 W).

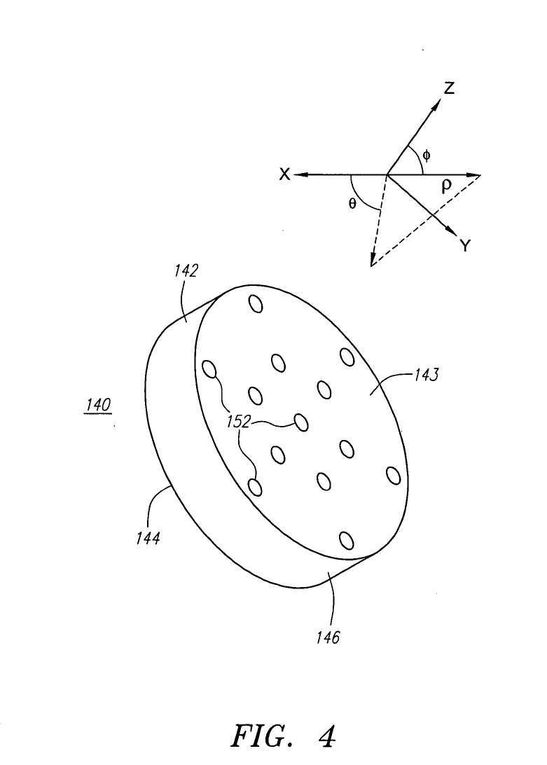

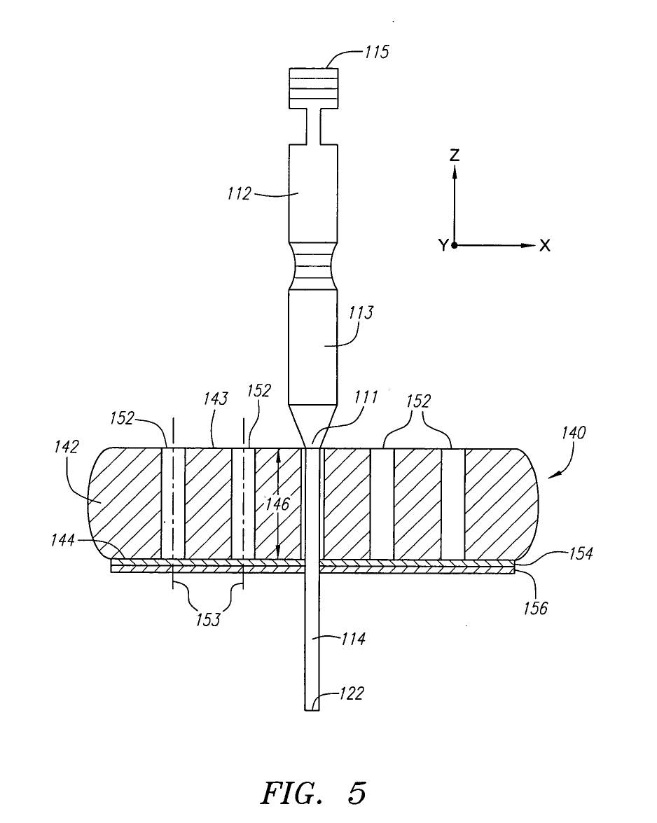

Referring specifically now to FIGS. 4 and 5, the alignment device 140 generally comprises a rigid base 142 having flat top and bottom surfaces 143 and 144 that are separated by a thickness 146. Although the rigid base 142 is disk-shaped in the illustrated embodiment, it can take on other shapes, such as rectangular, oval, triangular, or custom shaped, depending on the geometry of the tissue to be ablated. The size of the disk-shaped base 142 will ultimately depend at least in part on the volume of the tissue to be ablated.

The alignment device 140 further comprises a plurality of apertures 152 spaced along the top surface 143 of the base 142. The apertures 152 extend completely through the thickness 146 of the base 142, such that the apertures 152 are likewise also spaced along the bottom surface 144 of the base 152. In the illustrated embodiment, the apertures 152 are arranged in concentric rings around a center aperture. Depending upon the geometry of the tissue to be ablated and/or the geometry of the alignment structure, the apertures can be arranged in various other patterns.

As shown in FIG. 1, each aperture 152 is large enough, such that the cannula 114 of the probe assembly 110 can be passed through the alignment device 140, yet small enough, such that the distal handle member 113 of the handle assembly 112 cannot be passed through the alignment device 140. That is, each aperture 152 allows the cannula 114 to be passed through the alignment device 140 until the distal handle member 113 abuts the aperture 152, presumably when an interfering portion 111 of the distal handle member (i.e., the distal most portion of the handle member 113 having a diameter equal to the diameter of the aperture 152) coincides with the aperture 152. Preferably, the diameters of the cannula 114 and apertures 152 are closely toleranced, and the structure 142 is relatively thick, so that the cannula 114 remains aligned with the longitudinal axis of the particular aperture 152 as it passes therethrough. In this embodiment, as shown in FIG. 5, the axes 153 of the aperture 152 are parallel to each other.

Thus, it can be appreciated that the alignment device 140 can effectively align the distal end 122 of the cannula 114 within a two-dimensional Cartesian coordinate system, as it is passed through an aperture 152, with the two dimensions (x and y coordinates) being provided by the spacing between the apertures 152 on the flat top and bottom surfaces 143 and 144. To the extent that the cannula 114 can be inserted into the apertures 152 until the distal handle member 113 abuts the respective apertures 152, the alignment device 140 can effectively align the distal end 122 of the cannula 114 within a three-dimensional Cartesian coordinate system, with the third dimension (z coordinate) being provided by the top surface 143 of the base 142.

To the extent that spacings between the apertures are known, the alignment device 140 indexes the distal end 122 of the cannula 114 within a two-dimensional plane. In this embodiment, the apertures 152 are equally spaced to provide a consistent and easily usable indexing scheme. In this manner, ablation of the entire tumor will be assured by properly spacing the centers of the lesions created on the tumor. It can be appreciated that, in alternative embodiments, some or all of the apertures 152 may not be uniformly spaced.

In the preferred embodiment, the alignment device 140 is adhered directly to the patient although it is contemplated that other means for ensuring that the alignment device 140 remains fixed in relation to the target tissue can be utilized. For example, as shown in FIG. 5, the bottom surface 144 of the base 142 can be coated with a sticky substance 154 that is then covered with a substrate 156 that has a low affinity to the sticky substance 154. Prior to the operation, the substrate 156 can then be peeled off of the base 142 to expose the adhesive 154 on the respective surface of the substrate 156. As another example, the skin of the patient can be coated with a sticky substance. In either example, the alignment device 140 can then simply be adhered to the patient with very little pressure. Whichever method of adhesion is used, is preferable that it be temporary and not cause damage to the skin or other tissues while securing the alignment device 140 in a fixed position relative to the tumor.

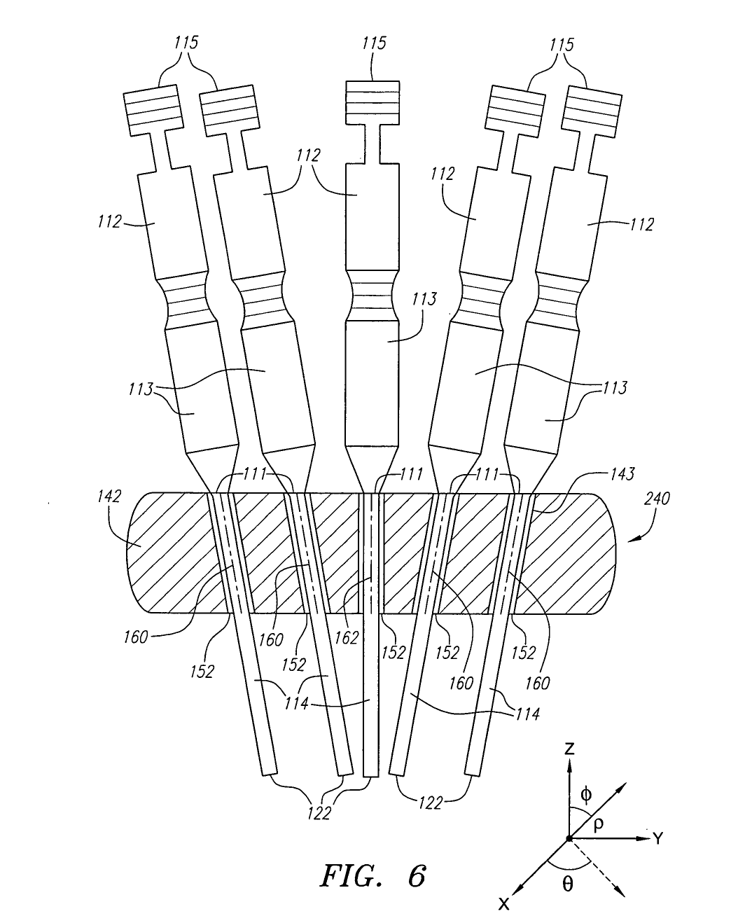

Referring now to FIG. 6, another alignment device 240 that can be used in the tissue ablation system 100 is described. The alignment device 240 is similar to the alignment device 140 illustrated in FIG. 5, with the exception that it comprises apertures 152 that have non-parallel axes 160. In particular, the axes 160 of the apertures 152 are angled towards a longitudinal axis 162 of the alignment device 140. Thus, it can be appreciated that the alignment device 240 can effectively align the distal end 122 of the cannula 114 within a three-dimensional angular coordinate system, with the two dimensions (angles φ and θ) being provided by the angles of the aperture axes 160. To the extent that the cannula 114 can be inserted into the apertures 152 until the distal handle member 113 abuts the respective apertures 152, the alignment device 140 can effectively align the distal end 122 of the cannula 114 within a three-dimensional spherical coordinate system, with the third dimension (radius p) being provided by the top surface 143 of the base 142.

The angles of the aperture axes 160 relative to the longitudinal axis 162 will depend upon the length of the cannula 114 (as dictated by depth of tumor) and the size of the tumor to be treated. For example, for a given tumor size, the angles of the axes 160 will be inversely proportional to the length of the cannula 114, so that the locations of the distal end 122 of the cannula 114 will be distributed about the entire tumor as it is inserted through each of the apertures 152.

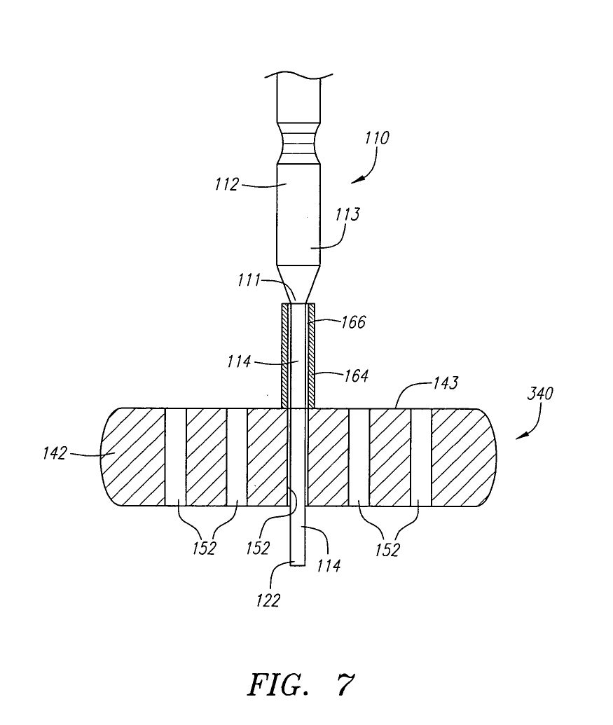

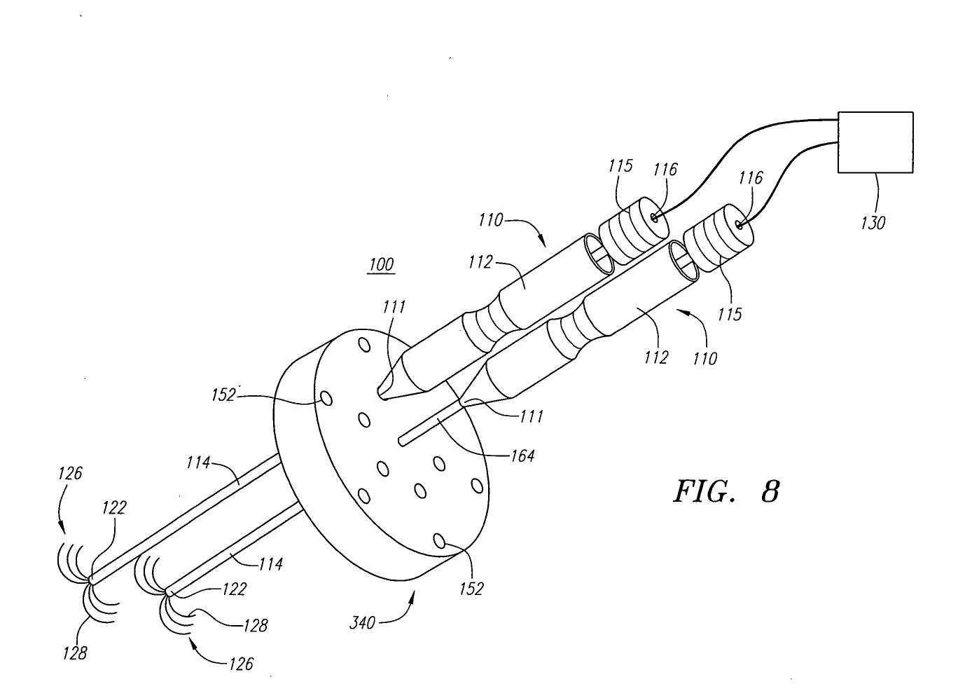

Referring now to FIG. 7, another alignment device 340 that can be used in the tissue ablation system 100 is shown. The alignment device 340 is similar to the previously described alignment device 140, with the exception that it comprises a single boss 164 mounted to the center aperture 152 of the base 142. The boss 164 prevents the distal end 122 of the cannula 114 to be guided to a lesser depth in the targeted tissue by offsetting the interfering portion 111 of the distal handle member 113 from the top surface 143 of the base 142. Specifically, the boss 164 comprises a cylindrical bore 166 that is sized to pass the cannula 114 of the probe assembly 110, yet causes the interfering portion 111 of the distal handle member 113 to abut against the boss 164, thereby limiting the distal movement of the cannula 114. In the preferred embodiment, the diameter of the bore 166 is equal to the diameter of the aperture 152. Thus, it can be appreciated that the alignment device 340, like the previously described alignment device 140, can effectively align the distal end 122 of the cannula 114 within a three-dimensional Cartesian coordinate system. The difference is that, to the extent that the height of the boss 164 is known, the alignment device 140 allows the distal end 122 of the cannula 114 to be indexed in three-dimensional space, rather than just a two-dimensional plane.

The boss 164 can be used with both monopolar and bipolar ablation techniques as described in more detail below, but are particularly useful in bipolar ablation to maintain the proper distance between two or more ablation probe assemblies 110, as illustrated in FIG. 8.

Referring again to FIG. 7, the boss 164 is permanently mounted to the center aperture 152. In other embodiments, the boss 164 may be removably mounted to the center aperture 152 using suitable means, such as a threaded arrangement. In this manner, the physician can customize the alignment device 140. For example, the physician can associate the boss 164 with another aperture 152, or completely remove the boss 164 from the base 142, so that the alignment device 140 indexes the distal end 122 of the cannula 114 within a two-dimensional plane, rather than three-dimensional space.

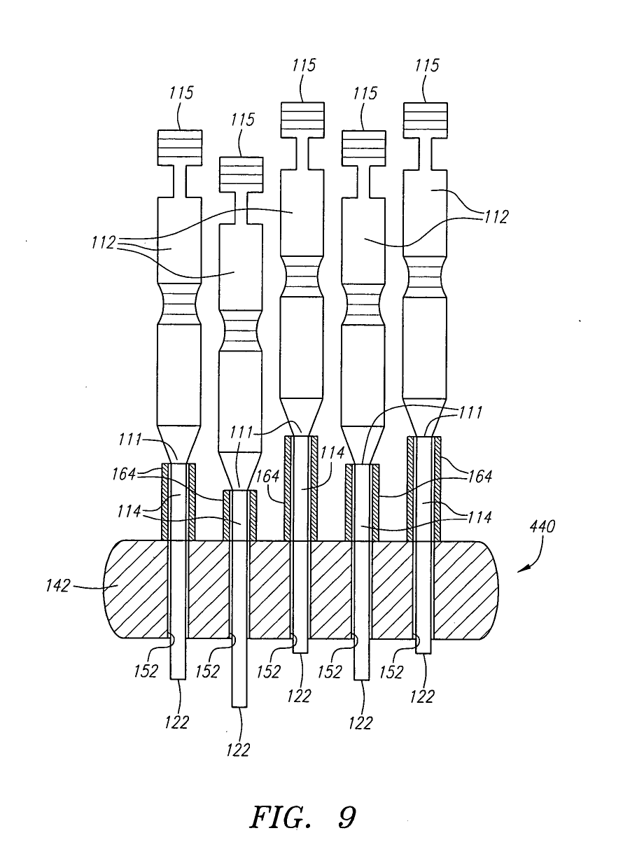

Although the alignment device 340 has a single boss 164 to index the distal end 122 of the cannula 114 at a different depth when it is fully inserted into the center aperture 152, a plurality of bosses 164 can be provided. For example, FIG. 9 illustrates an alignment device 440 that is similar to the previously described alignment device 340, with the exception that it includes a plurality of bosses 164 that are associated with a respective plurality of the apertures 152. In this manner, the alignment device 440 indexes the distal end 122 of the cannula 114 at a different depth when it is fully inserted into any one of apertures 152 with which a boss 164 is associated. As shown, the bosses 164 have different heights, so that the alignment device 140 can index the distal end 122 of the cannula 114 at a variety of depths.

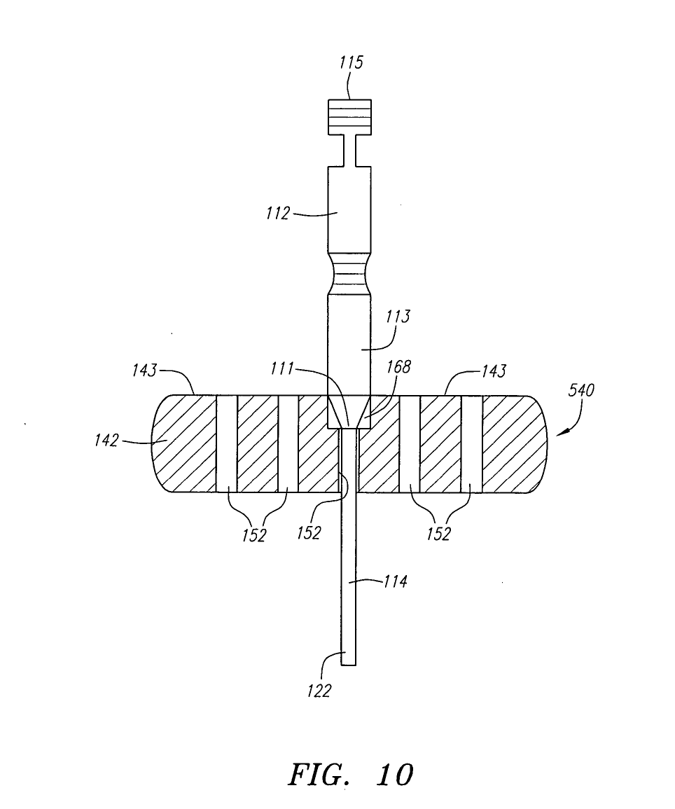

The use of bosses is not the only way to index the distal end 122 of the cannula 114 at different depths. For example, referring to FIG. 10, another alignment device 540 that can be used in the tissue ablation system 100 is shown. The alignment device 540 is similar to the previously described alignment device 340, with the exception that it comprises a single recess 168 (rather than a boss) formed within the center aperture 152. The recess 168 allows the distal end 122 of the cannula 114 to be guided to a greater depth in the targeted tissue by allowing the interfering portion 111 of the distal handle member 113 to extend below the top surface 143 of the base 142. Specifically, the recess 168 is sized to pass the interfering portion 111 of the distal handle member 113, so that it abuts against the center aperture 152 below the top surface 143 of the base 142, thereby extending the distal movement of the cannula 114. Thus, to the extent that the depth of the recess 168 is known, the alignment device 140, like the previously described alignment device 140, allows the distal end 122 of the cannula 114 to be indexed in three-dimensional space.

Like the boss 164, the recess 168 can be used with both monopolar and bipolar ablation techniques as described in more detail below, but is particularly useful in bipolar ablation to maintain the proper distance between two or more ablation probe assemblies 110, as illustrated in FIG. 8.

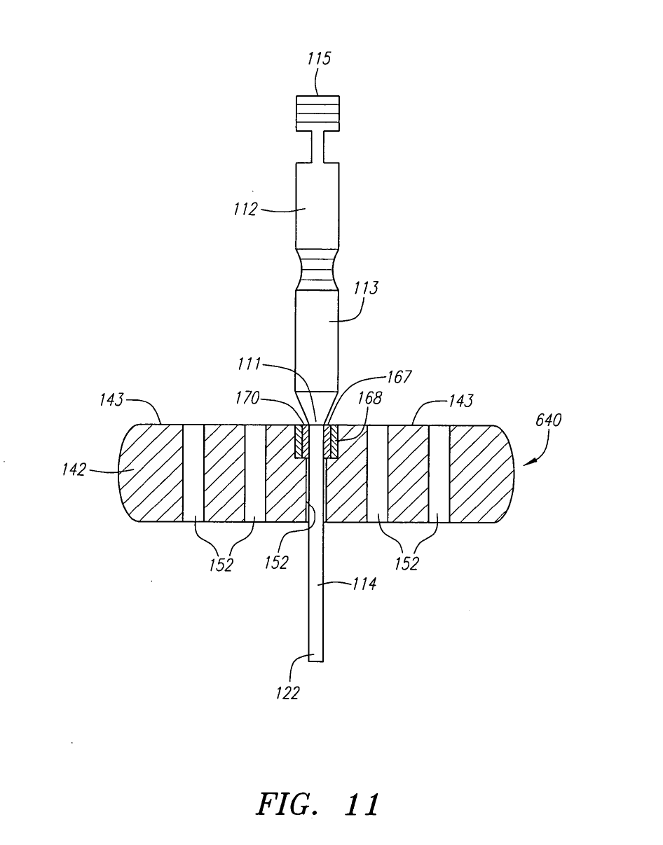

As illustrated in FIG. 11, an alignment device 640 similar to the alignment device 540 may optionally comprise an insert 170 that is removably mounted within the center aperture 152 using suitable means, such as a threaded arrangement. The insert 170 is cylindrical, although it is contemplated that it could be another shape such as square or rectangular, and has a bore 167 that is aligned with the central aperture 152. The bore 166 is sized to pass the cannula 114 of the probe assembly 110, yet cause the interfering portion 111 of the distal handle member 113 to abut against the insert 170, thereby limiting the distal movement of the cannula 114. In the preferred embodiment, the diameter of the bore 166 is equal to the diameter of the aperture 152. Thus, the insert 170 functions to fill in the recess 168 of the center aperture 152, such that the center aperture 152 functions as an aperture 152 with no recess.

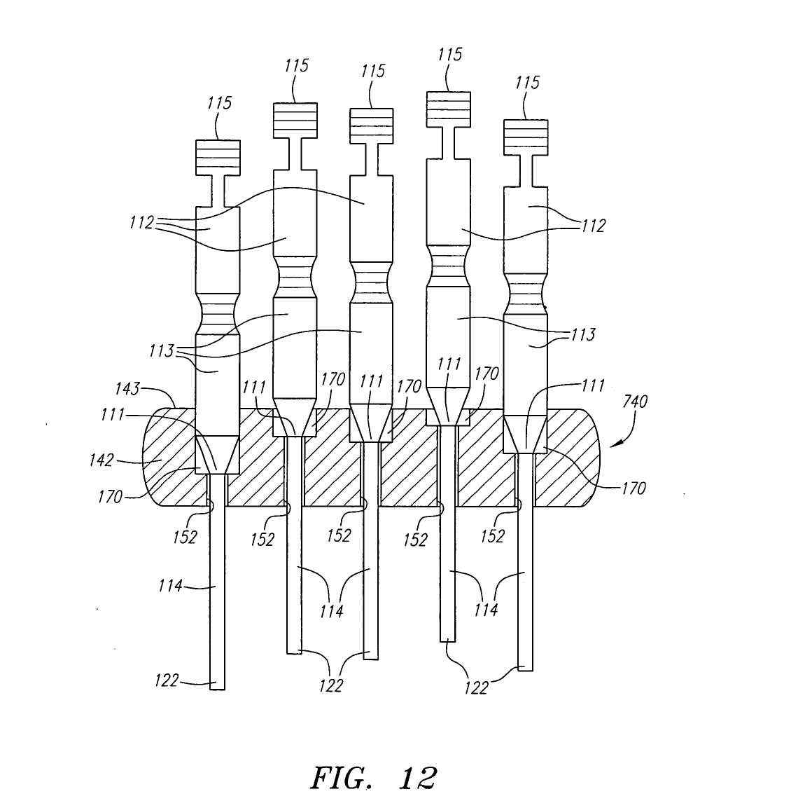

Although the alignment device 440 illustrated in FIG. 10 has a single recess 168 to index the distal end 122 of the cannula 114 at a different depth when it is fully inserted into the center aperture 152, a plurality of recesses 168 can be provided. For example, FIG. 12 illustrates an alignment device 740 that is similar to the previously described alignment device 540, with the exception that it includes a plurality of recesses 168 that are associated with a respective plurality of the apertures 152. In this manner, the alignment device 740 indexes the distal end 122 of the cannula 114 at a different depth when it is fully inserted into any one of apertures 152 with which a recess 168 is associated. As illustrated in FIG. 12, the recesses 168 have different depths, so that the alignment device 740 can index the distal end 122 of the cannula 114 at a variety of depths. The alignment device 740 can be customized by providing inserts (shown in FIG. 11) that can be selectively inserted into the recesses 168. The inserts can have different heights, so that the alignment device 140 can index the distal end 122 of the cannula 114 at a variety of depths.

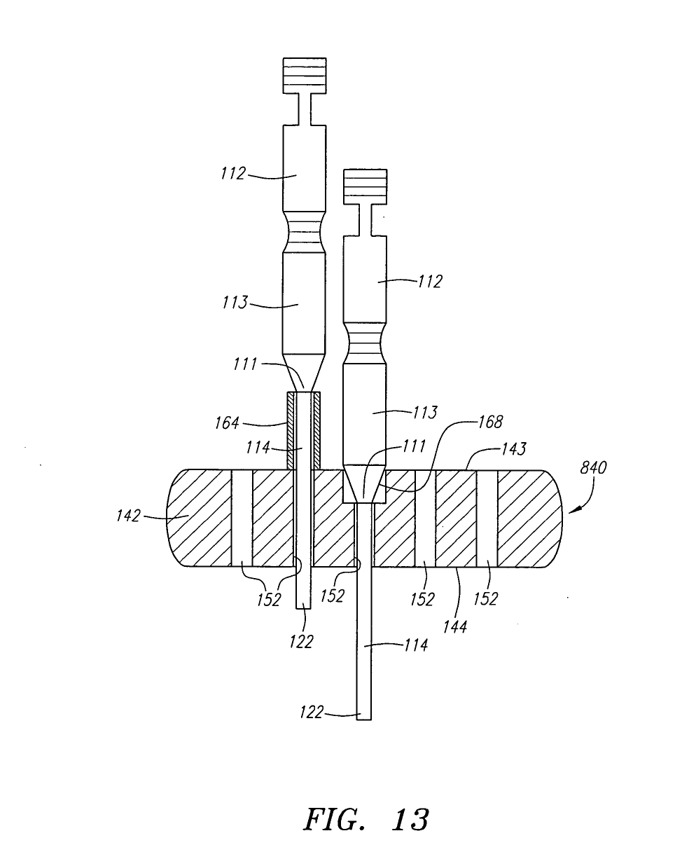

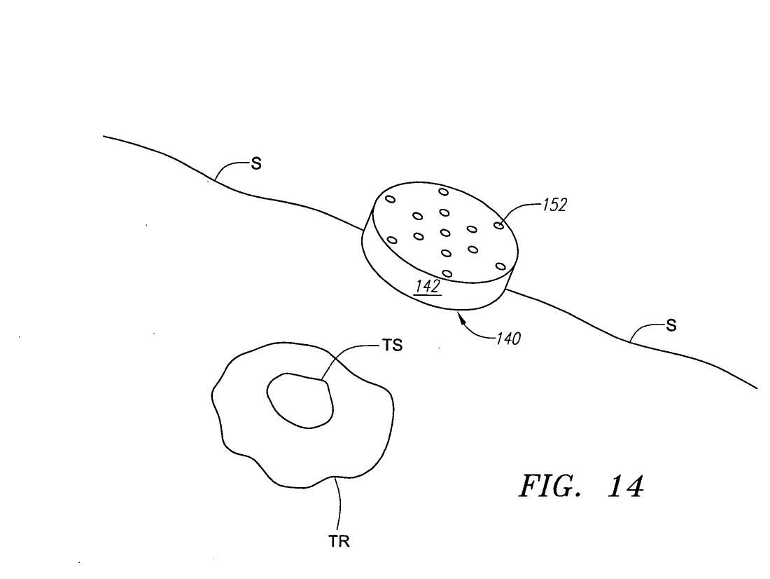

In further alternative embodiments, an alignment device 840 can have both bosses 164 and recesses 168, as illustrated in FIG. 13, so that the interfering portion 111 of the distal handle member 113 can be offset from the top surface 143 of the base 142 or extend below the top surface 143 of the base 142. In this manner, the distal end 122 of the cannula 114 can be indexed at a greater range of depths.

Having described the structure of the tissue ablation system 100, its operation in treated targeted tissue will now be described. The treatment region may be located anywhere in the body where hyperthermic exposure may be beneficial. Most commonly, the treatment region will comprise a solid tumor within an organ of the body, such as the liver, kidney, pancreas, breast, prostrate (not accessible via the urethra), and the like. The volume to be treated will depend on the size of the tumor or other lesion, typically having a total volume from 1 cm³ to 150 cm³, and often from 2 cm³ to 35 cm³. The peripheral dimensions of the treatment region may be regular, e.g., spherical or ellipsoidal, but will more usually be irregular. The treatment region may be identified using conventional imaging techniques capable of elucidating a target tissue, e.g., tumor tissue, such as ultrasonic scanning, magnetic resonance imaging (MRI), computer assisted tomography (CAT) fluoroscopy, nuclear scanning (using radiolabeled tumor-specific probes), and the like. Preferred is the use of high resolution ultrasound of the tumor or other lesion being treated, either intraoperatively or externally. The image of the tumor is used to determine where the alignment device 140 should be fixed in order to introduce the cannula 114 and inner probe 118 to the target site. It can also be appreciated that a plan for conducting multiple ablations on the tumor can be mapped out prior to the procedure using the image of the tumor and the alignment device 140.

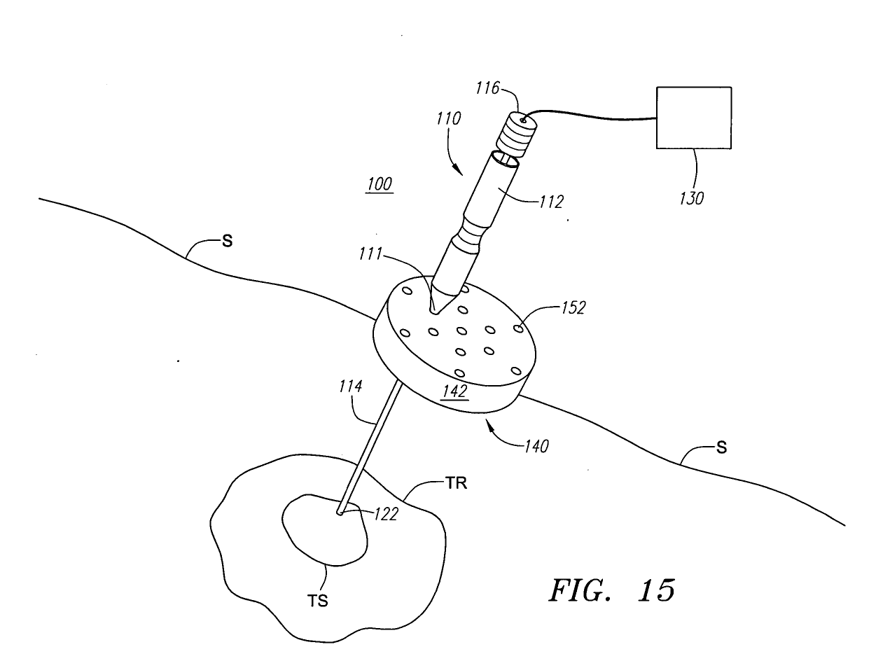

Referring now to FIGS. 14-17, the operation of the tissue ablation system 100 is described in treating a treatment region TR, such as a tumor, located beneath the skin S of a patient. First, the alignment device 140 is affixed relative to the targeted tissue, as illustrated in FIG. 14. In the preferred embodiment, the alignment device 140 is adhered directly to the skin of the patient by, e.g., peeling the substrate 156 off of the bottom surface 144 of the base 142, and pressing the base 142 against the body of the patient 172. It is contemplated that other means of adhesion may be used.

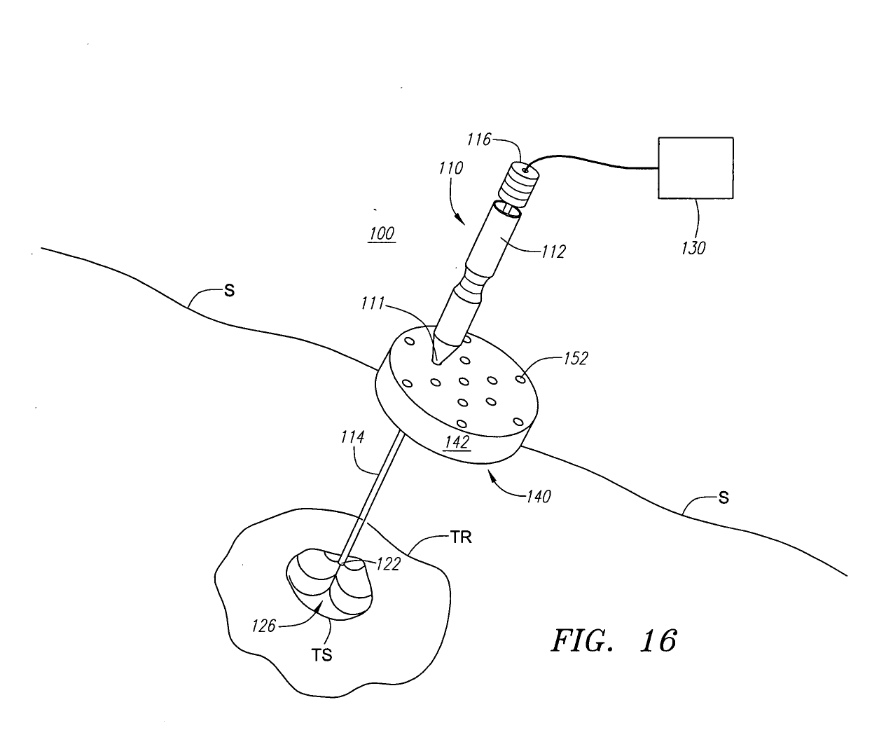

The cannula 114 of the probe assembly 110 is then guided within an aperture 152 of the alignment device 140, as illustrated in FIG. 15. The cannula 114 passes through the aperture 152 of the alignment device 140 until its distal end 122 is adjacent a first target site TS1 within the tumor T. The cannula 114 and inner probe 118 may be introduced into the first target site TS1 percutaneously—i.e., directly through the patient's skin—or through an open surgical incision. If the cannula 114 is introduced through an open surgical incision, the incision will be made prior to fixing the alignment device 140 relative to the treatment region TR. In this case, the alignment device 140 will span the open incision. When the introduction is done percutaneously, the cannula 114 may have a sharpened tip like a needle, to facilitate introduction into the treatment region TR. In this case, it is desirable that the cannula 114 be sufficiently rigid, i.e., have a sufficient columnar strength, so that it can be accurately advanced through the surrounding volume of tissue. Alternatively, the cannula 114 may be introduced using an internal stylet that is subsequently exchanged for the shaft 121 and electrode array 126. In this latter case, the cannula 114 can be relatively flexible, since the initial columnar strength will be provided by the stylet.

After the cannula 114 is properly placed so that its distal end 122 is adjacent to the first target site TS 1, the shaft 121 is distally advanced to deploy the electrode array 126 radially outward from the distal end 122 of the cannula 114, as illustrated in FIG. 16. The shaft 121 is advanced sufficiently, so that the electrode array 126 fully everts in order to substantially penetrate the first treatment site TS 1. If the probe assembly 110 has an optional core member (not shown) previously mentioned, then the sharpened end of the core member facilitates introduction of the electrode array 126 into the treatment region. The RF generator 130 is then connected to the ablation probe assembly 110 via the electrical connector 116, and then operated to ablate the treatment region resulting in the formation of a lesion that is coincident with the first treatment site TS1.

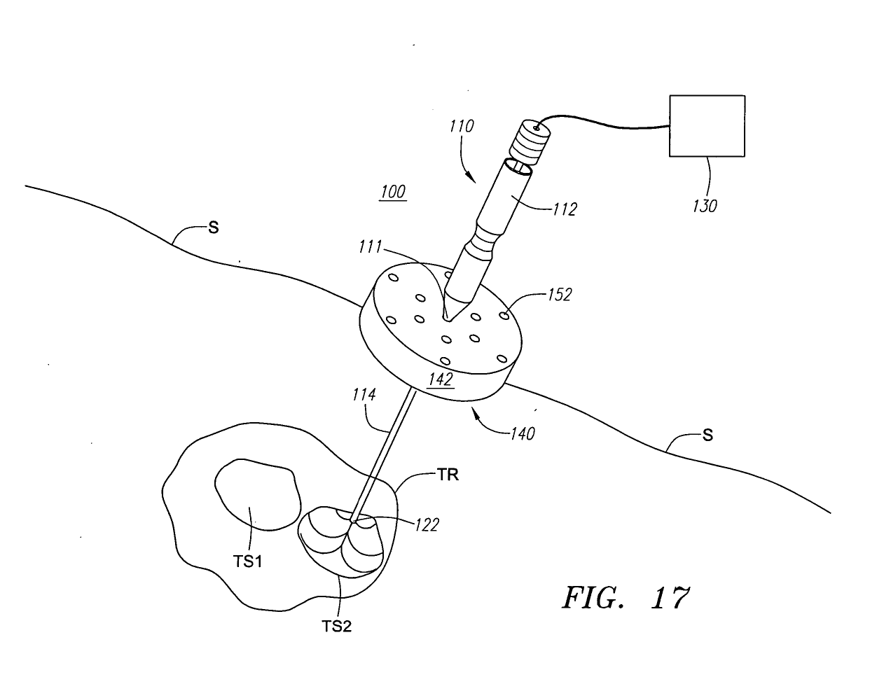

Referring to FIG. 17, the ablation probe assembly 110 is removed from the first aperture 152, and then guided through a second different aperture 152 in the alignment device 140 to place the distal end 122 of the cannula 114 adjacent to the targeted tissue in a second target site TS2 within the treatment region TR. The RF generator 130 is then operated a second time to create a second lesion that encompasses the second target site TS2. This process is performed using other apertures 152 until the entire treatment region TR is ablated. Thus, it can be appreciated that, by using the alignment device 140, the distal end 122 of the cannula 114 is indexed with a two-dimensional plane that extends through the treatment region TR.

In an optional method, lesions can be created within the treatment region TR at multiple depths, by retracting the electrode array 126 within the cannula 114 after performing an ablation, and adjusting the cannula 114 within the same aperture 152 so that its distal end 122 is adjacent another treatment site that is spaced from the first treatment site TS1 along the axis 160 of the aperture 152. The electrode array 126 is then deployed within the other treatment site, and the RF generator 130 is operated another time to create a second lesion that encompasses the other target site. This step can be repeated for the same aperture to generate lesions at various depths, and can be repeated for other apertures. This optional step is particularly useful if the depth of the treatment region TR is greater than the depth of a single lesion that can be created by the probe assembly 110. If indexing of the various depths are desired, any one of the alignment devices 340-840 can be used to index the distal end 122 of the cannula 114 within the three-dimensional space occupied by the treatment region TR.

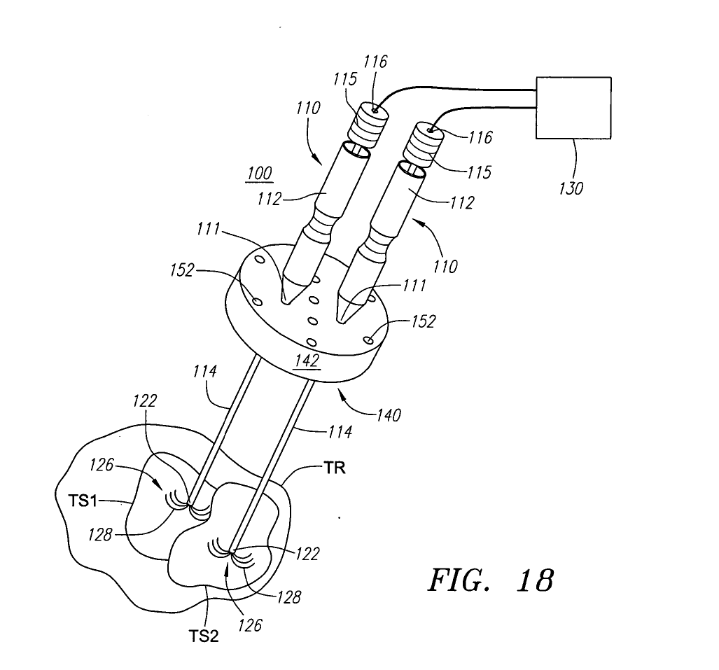

In another preferred method, a plurality of ablation probe assemblies 110 may be guided through a respective plurality of apertures 152 in the alignment device 140 to place the distal ends 120 of the cannula 114 adjacent to multiple target sites TS of the tissue, and then the respective electrode arrays 126 can then be deployed from the distal ends 122 of the cannula 114, as illustrated in FIG. 18. In a unipolar mode, the RF generator 130 can be operated to sequentially generate lesions from the probe assemblies 110 within the target region TR. In a bipolar mode, the RF generator 130 can operate pairs of probe assemblies 110 to generate lesions between the probe assemblies 110 by conveying RF energy between the respective electrode arrays 126. For example, the probe assembly 110 associated with center aperture 152 can be sequentially paired with the remaining probe assemblies 110 to generate lesions between the center electrode array 126 and the remaining electrode arrays 126.

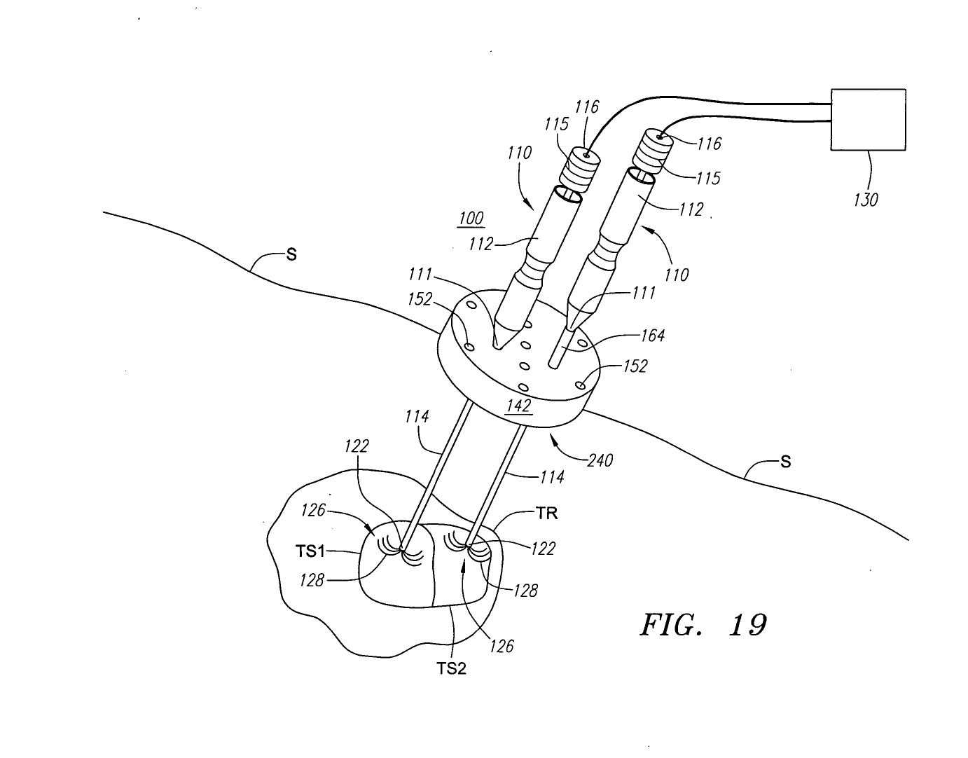

As illustrated in FIG. 19, the alignment device 240 can be used to offset the center electrode array 126 a predetermined distance from the remaining electrode arrays 126. In this manner, the proper distance is maintained between the electrode arrays 126 to efficiently produce a lesion there between. One skilled in the art would appreciate that the needle electrodes 128 from the different ablation probe assemblies 110 should be insulated from touching the needle electrodes 128 from the other ablation probe assemblies 110. This process may be repeated or a sufficient number of ablation probe assemblies may be used such that the entire target region is ablated.

If indexing of the various depths are desired, any one of the alignment devices 340-840 can be used to index the distal ends 122 of the cannulae 114 within the three-dimensional space occupied by the treatment region TR.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A tissue ablation system, comprising:
one or more ablation probes;
an alignment device configured to be fixed relative to targeted tissue, the alignment device comprising a surface and a plurality of apertures through which the one or more ablation probes can be guided, the plurality of apertures spaced apart along the surface.

2. The system of claim 1, wherein the alignment device is configured to be adhered to a patient.

3. The system of claim 1, wherein the alignment device is disk-shaped.

4. The system of claim 1, wherein the spacing between the apertures is fixed.

5. The system of claim 1, wherein the spacing between the apertures is adjustable.

6. The system of claim 1, wherein the spacing between the apertures is uniform.

7. The system of claim 1, wherein the apertures are indexed from each other in a two-dimensional plane.

8. The system of claim 1, wherein the apertures are indexed from each other in three-dimensional space.

9. The system of claim 1, wherein the plurality of apertures comprises a central aperture and remaining apertures that are placed in a plurality of concentric rings around the central aperture.

10. The system of claim 1, wherein the apertures have axes that are parallel to each other.

11. The system of claim 1, wherein the apertures have axes that are non-parallel to each other.

12. The system of claim 1, wherein the surface is flat.

13. The system of claim 1, wherein the alignment device comprises one or more bosses associated with a respective one or more of the plurality of apertures, wherein the one or more bosses limits the distance that the one or more ablation probes can be guided through the one or more apertures.

14. The system of claim 13, wherein the one or more bosses is removably mounted to the one or more apertures.

15. The system of claim 13, wherein the one or more bosses is permanently mounted to the one or more apertures.

16. The system of claim 13, wherein the one or more bosses comprises a plurality of bosses.

17. The system of claim 16, wherein the bosses have differing lengths.

18. The system of claim 1, wherein the alignment device comprises one or more recesses associated with a respective one or more of the plurality of apertures, wherein the one or more recesses extends the distance that the one or more ablation probes can be guided through the one or more apertures.

19. The system of claim 18, wherein the recesses have differing depths.

20. The system of claim 18, wherein the alignment device comprises one or more inserts associated with a respective one or more recesses, wherein the insert is removably mounted.

21. The system of claim 1, wherein each of the one or more ablation probes is a radio frequency (RF) ablation probe.

22. The system of claim 1, wherein the one or more ablation probes comprises a plurality of ablation probes.

23. A method for performing a compound ablation in the body of a patient, comprising:
affixing an alignment device relative to targeted tissue;
guiding an ablation probe within a first aperture in the alignment device to place the ablation probe adjacent the targeted tissue in a first region;
operating the ablation probe to create a first lesion in the first region;
guiding the ablation probe within a second different aperture in the alignment device to place the ablation probe adjacent the targeted tissue in a second region; and
operating the ablation probe again to create a second lesion in the second region.

24. The method of claim 23, further comprising completely removing the ablation probe from the first aperture prior to guiding the first ablation probe within the second aperture.

25. The method of claim 23, wherein alternate guiding and operating of the ablation probe is performed for a plurality of regions until the entire target tissue is ablated.

26. The method of claim 23, wherein the ablation probe is guided within the first and second apertures in parallel directions.

27. The method of claim 23, wherein the ablation probe is guided within the first and second apertures in non-parallel directions.

28. The method of claim 23, wherein the alignment device comprises a boss or a recess associated within the first aperture, the method further comprising modifying a distance that the ablation probe is guided within the first aperture by abutting a portion of the ablation probe against the boss or recess.

29. The method of claim 23, wherein the ablation probe is operated by generating RF energy to create the first and second lesions.

30. The method of claim 23, wherein the ablation probe is placed in contact with the first and second regions of the target tissue.

31. The method of claim 23, wherein the ablation probe is embedded with the first and second regions of the target tissue.

32. The method of claim 23, wherein the target tissue is inside the body of the patient.

33. The method of claim 23, wherein the ablation probe is percutaneously guided within the first and second apertures into the body of the patient.

34. The method of claim 23, wherein the target tissue is a tumor.

35. A method for performing a compound ablation in the body of a patient, comprising:
affixing an alignment device relative to targeted tissue;
guiding a plurality of ablation probes within a respective plurality of apertures in the alignment device to place the ablation probes adjacent the targeted tissue in a plurality of regions;
operating the ablation probes to create a plurality of lesions in the plurality of regions.

36. The method of claim 35, wherein the plurality of ablation probes are operated by transmitting RF energy between at least two of the ablation probes.

37. The method of claim 35, wherein the entire target tissue is ablated.

38. The method of claim 35, wherein the ablation probes are guided within the plurality of apertures in parallel directions.

39. The method of claim 35, wherein the ablation probes are guided within the plurality of apertures in non-parallel directions.

40. The method of claim 35, wherein the alignment device comprises one or more bosses or recesses associated within one or more of the plurality of apertures, the method further comprising modifying a distance that one or more of the ablation probes are guided within the one or more plurality of apertures by abutting a portion of the one or more ablation probes against the one or more bosses or recesses.

41. The method of claim 40, wherein the one or more bosses comprises a plurality of bosses.

42. The method of claim 41, wherein the bosses have differing lengths.

43. The method of claim 40, wherein one or more apertures is associated with one or more inserts, wherein one or more inserts are removably mounted.

44. The method of claim 35, wherein the ablation probes are operated by generating RF energy to create the plurality of lesions.

45. The method of claim 35, wherein the ablation probes are placed in contact with the plurality of regions of the target tissue.

46. The method of claim 35, wherein the ablation probes are embedded with the plurality of regions of the target tissue.

47. The method of claim 35, wherein the target tissue is inside the body of the patient.

48. The method of claim 47, wherein the ablation probes are percutaneously guided within the plurality of apertures into the body of the patient.

49. The method of claim 35, wherein the target tissue is a tumor.

50. An alignment device for one or more ablation probes, comprising:
a surface;
a plurality of apertures, through which the one or more ablation probes can be guided, wherein the plurality of apertures are spaced apart along the surface; and
one or more bosses or recesses associated with a respective one or more of the plurality of apertures, wherein the one or more bosses or recesses modifies the distance that the one or more ablation probes can be guided through the one or more apertures.

51. The device of claim 50, wherein the alignment device is configured to be adhered to a patient.

52. The device of claim 50, wherein the alignment device is disk-shaped.

53. The device of claim 50, wherein the spacing between the apertures is fixed.

54. The device of claim 50, wherein the spacing between the apertures is adjustable.

55. The device of claim 50, wherein the spacing between the apertures is uniform.

56. The device of claim 50, wherein the apertures are indexed from each other in a two-dimensional plane.

57. The device of claim 50, wherein the apertures are indexed from each other in three-dimensional space.

58. The device of claim 50, wherein the plurality of apertures comprises a central aperture and remaining apertures that are placed in a plurality of concentric rings around the central aperture.

59. The device of claim 50, wherein the apertures have axes that are parallel to each other.

60. The device of claim 50, wherein the apertures have axes that are non-parallel to each other.

61. The device of claim 50, wherein the surface is flat.

62. The device of claim 50, wherein the one or more bosses or recesses comprises one or more bosses that limit the distance that the one or more ablation probes can be guided through the one or more apertures.

63. The device of claim 50, wherein the one or more bosses is removably mounted to the one or more apertures.

64. The device of claim 50, wherein the one or more bosses is permanently mounted to the one or more apertures.

65. The device of claim 50, wherein the one or more bosses or recesses comprises one or more recesses that extend the distance that the one or more ablation probes can be guided through the one or more apertures.

66. The device of claim 50, wherein the one or more bosses or recesses comprises one or more bosses and one or more recesses.

67. The device of claim 50, wherein the one or more bosses or recesses comprises a plurality of bosses or recesses.

68. The device of claim 67, wherein the plurality of bosses or recesses have differing lengths.

69. The device of claim 50, wherein the one or more recesses is associated with one or more inserts removably mounted in the associated one or more recesses.
